(19) United States
(12) Patent Application Publication
(10) Pub. No.: US 2007/0106132 A1
(43) Pub. Date: May 10, 2007

(54) Monitoring device, method and system

(76) Inventors: Sammy I. Elhag, San Diego, CA (US); Donald Brady, Las Vegas, NV (US); Steve Lui, San Diego, CA (US)

Correspondence Address: IMPACT SPORTS TECHNOLOGIES, INC., 7574 Trad Street, Suite B, San Diego, CA, 92121, US

(21) Appl. No.: 11/388,707

(22) Filed: Mar. 24, 2006

Related U.S. Application Data
(63) Continuation-in-part of application No. 11/085,778, filed on Mar. 21, 2005.
(60) Provisional application No. 60/665,116, filed on Mar. 25, 2005.
(60) Provisional application No. 60/613,785, filed on Sep. 28, 2004.

(51) Int. Cl. A61B 5/00; A61B 5/02

(52) U.S. Cl. 600/301; 600/323; 600/504; 600/500; 128/920

(57) ABSTRACT

A monitoring device (20) and system (50) for monitoring the vital signs of a user is disclosed herein. The monitoring device (20) is preferably an article (25), an optical sensor (30) and a circuitry assembly (35). The monitoring device (20) preferably provides for the display of the following information about the user: pulse rate; blood oxygenation levels; calories expended by the user of a pre-set time period; target zones of activity; time; distance traveled; and dynamic blood pressure. The article (25) is preferably a band worn on a user's wrist, arm or ankle. The system (50) allows for real-time monitoring and display of the vital signs of an athlete or player during a live event.

### CROSS REFERENCES TO RELATED APPLICATION

The Present Application claims priority to U.S. Provisional Patent Application No. 60/665,116, filed on Mar. 25, 2005. The Present Application is also a continuation-in-part application of U.S. patent application Ser. No. 11/085,778, filed on Mar. 21, 2005, which is a continuation-in-part application of U.S. Provisional Application No. 60/613,785, filed on Sep. 28, 2004, now abandoned.

### STATEMENT REGARDING FEDERALLY SPONSORED RESEARCH OR DEVELOPMENT

Not Applicable

### BACKGROUND OF THE INVENTION

1. Field of the Invention

The present invention is related to real-time vital sign monitoring devices. More specifically, the present invention relates to a wrist, arm or ankle band for monitoring a user's vital signs, and a system that uses the monitoring device to display a real-time vital sign of an athlete/player during an event.

2. Description of the Related Art

There is a need to know how one is doing from a health perspective. In some individuals, there is a daily, even hourly, need to know one's health. The prior art has provided some devices to meet this need.

One such device is a pulse oximetry device. Pulse oximetry is used to determine the oxygen saturation of arterial blood. Pulse oximeter devices typically contain two light emitting diodes: one in the red band of light (660 nanometers) and one in the infrared band of light (940 nanometers). Oxyhemoglobin absorbs infrared light while deoxyhemoglobin absorbs visible red light. Pulse oximeter devices also contain sensors that detect the ratio of red/infrared absorption several hundred times per second. A preferred algorithm for calculating the absorption is derived from the Beer-Lambert Law, which determines the transmitted light from the incident light multiplied by the exponential of the negative of the product of the distance through the medium, the concentration of the solute and the extinction coefficient of the solute.

The major advantages of pulse oximetry devices include the fact that the devices are non-invasive, easy to use, allows for continuous monitoring, permits early detection of desaturation and is relatively inexpensive. The disadvantages of pulse oximetry devices are that it is prone to artifact, it is inaccurate at saturation levels below 70%, and there is a minimal risk of burns in poor perfusion states. Several factors can cause inaccurate readings using pulse oximetry including ambient light, deep skin pigment, excessive motion, fingernail polish, low flow caused by cardiac bypass, hypotension, vasoconstriction, and the like.

Chin et al., U.S. Pat. No. 6,018,673 discloses a pulse oximetry device that is positioned entirely on a user's nail to reduce out of phase motion signals for red and infrared wavelengths for use in a least squares or ratio-of-ratios technique to determine a patient's arterial oxygen saturation.

Smith, U.S. Pat. No. 4,800,495 discloses an apparatus for processing signals containing information concerning the pulse rate and the arterial oxygen saturation of a patient. Smith also discloses maintaining the position of the LEDs and detectors to prevent motion-artifacts from being produced in the signal.

Another method for using a pulse oximeter to measure blood pressure is disclosed in U.S. Pat. No. 6,616,613 to Goodman for a ‘Physiological Signal Monitoring System’. The '613 Patent discloses processing a pulse oximetry signal in combination with information from a calibrating device to determine a patient's blood pressure.

Chen et al, U.S. Pat. No. 6,599,251 discloses a system and method for monitoring blood pressure by detecting pulse signals at two different locations on a subjects body, preferably on the subject's finger and earlobe. The pulse signals are preferably detected using pulse oximetry devices.

Schulze et al., U.S. Pat. No. 6,556,852, discloses the use of an earpiece having a pulse oximetry device and thermopile to monitor and measure physiological variables of a user.

Malinouskas, U.S. Pat. No. 4,807,630, discloses a method for exposing a patient's extremity, such as a finger, to light of two wavelengths and detecting the absorbance of the extremity at each of the wavelengths.

Jobsis et al., U.S. Pat. No. 4,380,240 discloses an optical probe with a light source and a light detector incorporated into channels within a deformable mounting structure which is adhered to a strap. The light source and the light detector are secured to the patient's body by adhesive tapes and pressure induced by closing the strap around a portion of the body.

Tan et al, U.S. Pat. No. 4,825,879 discloses an optical probe with a T-shaped wrap having a vertical stem and a horizontal cross bar, which is utilized to secure a light source and an optical sensor in optical contact with a finger. A metallic material is utilized to reflect heat back to the patient's body and to provide opacity to interfering ambient light. The sensor is secured to the patient's body using an adhesive or hook and loop material.

Modgil et al., U.S. Pat. No. 6,681,454 discloses a strap that is composed of an elastic material that wraps around the outside of an oximeter probe and is secured to the oximeter probe by attachment mechanisms such as Velcro, which allows for adjustment after initial application without producing excessive stress on the spring hinge of the oximeter probe.

Diab et al., U.S. Pat. No. 6,813,511 discloses a disposable optical probe suited to reduce noise in measurements, which is adhesively secured to a patient's finger, toe, forehead, earlobe or lip.

Diab et al., U.S. Pat. No. 6,678,543 discloses an oximeter sensor system that has a reusable portion and a disposable portion. A method for precalibrating a light sensor of the oximeter sensor system is also disclosed.

Tripp, Jr. et al., U.S. Statutory Invention Registration Number H1039 discloses an intrusion free physiological condition monitor that utilizes pulse oximetry devices.

Hisano et al., U.S. Pat. No. 6,808,473, discloses a headphone-type exercise aid which detects a pulse wave using an optical sensor to provide a user with an optimal exercise intensity.

In monitoring one's health there is a constant need to know how many calories have been expended whether exercising or going about one's daily routine. A calorie is a measure of heat, generated when energy is produced in our bodies. The amount of calories burned during exercise is a measure of the total amount of energy used during a workout. This can be important, since increased energy usage through exercise helps reduce body fat. There are several means to measure this expenditure of energy. To calculate the calories burned during exercise one multiplies the intensity level of the exercise by one's body weight (in kilograms). This provides the amount of calories burned in an hour. A unit of measurement called a MET is used to rate the intensity of an exercise. One MET is equal to the amount of energy expended at rest.

For example, the intensity of walking 3 miles per hour (“mph”) is about 3.3 METS. At this speed, a person who weighs 132 pounds (60 kilograms) will burn about 200 calories per hour (60×3.3=198).

The computer controls in higher-quality exercise equipment can provide a calculation of how many calories are burned by an individual using the equipment. Based on the workload, the computer controls of the equipment calculate exercise intensity and calories burned according to established formulae.

The readings provided by equipment are only accurate if one is able to input one's body weight. If the machine does not allow this, then the “calories per hour” or “calories used” displays are only approximations. The machines have built-in standard weights (usually 174 pounds) that are used when there is no specific user weight.

There are devices that utilize a watch-type monitor to provide the wearer with heart rate as measured by a heartbeat sensor in a chest belt.

The prior art has failed to provide a means for monitoring one's health that is accurate, easy to wear on one's body for extended time periods, allows the user to input information and control the output, and provides sufficient information to the user about the user's health. Thus, there is a need for a monitoring device that can be worn for an extended period and provide health information to a user.

Further, the viewers of an athletic event have always desired to have information on the athletes' performance during the event. Recently, graphical displays during televised sporting events have included: the trajectory of a hockey puck during a hockey game; the first down line during a football game; the location of birdies, bogeys and eagles for a hole during a golf tournament; and the out of bounds line during a tennis match. Further, informational displays during televised sporting events have included: the speed of a pitch during a baseball game; the speed of a serve during a tennis match; the distance of a drive during a golf tournament; the money won and cards held by a poker player; and the number of punches thrown during a boxing match.

The modern viewer of athletic events demands a greater quantity of information for the performance of the athlete. The modern viewer wants sufficient information of the athlete/player to vicariously participate in the event through the athlete/player.

### BRIEF SUMMARY OF THE INVENTION

The present invention provides a solution to the shortcomings of the prior art. The present invention is accurate, comfortable to wear by a user for extended time periods, allows for input and controlled output by the user, is light weight, and provides sufficient real-time information to the user about the user's health. Further, the present invention allows for the transmission of a multitude of the user's real time vital signs to a video display monitor during an athletic event to enhance the viewing enjoyment of the audience.

One aspect of the present invention is a monitoring device for monitoring the health of a user. The monitoring device includes an article, an optical device for generating a pulse waveform, a circuitry assembly embedded within the article, a display member positioned on an exterior surface of the article, and a control means attached to the article.

The article is preferably a band to be worn on a user's wrist or ankle. The article preferably has a minimal mass, one to five ounces, and is flexible so that the user can wear it the entire day if necessary. The monitoring device allows the user to track calories burnt during a set time period, monitor heart rate, blood oxygenation levels, distance traveled, target zones and optionally dynamic blood pressure.

Another aspect of the present invention is a method for monitoring a user's vital signs. The method includes generating a signal corresponding to the flow of blood through an artery of the user. The signal is generated from an optical device. Next, the heart rate data of the user and an oxygen saturation level data of the user is generated from the signal. Next, the heart rate data of the user and the oxygen saturation level data of the user are processed for analysis of calories expended by the user and for display of the user's heart rate and blood oxygen saturation level. Next, the calories expended by the user, the user's heart rate or the user's blood oxygen saturation level are displayed on a display member on an exterior surface of an article, which is controlled by the user using a control component extending from the article.

Yet another aspect of the present invention is a system for real time monitoring of a user's vital sign during a live event within a playing environment. The system includes a monitoring device, a computing device and an electro-optical display. The monitoring device is attached to an arm, wrist or ankle of the user. The monitoring device comprises means for generating a real-time vital sign signal corresponding to the heart rate of the user, and means for transmitting the real-time vital sign signal outside of the playing environment. The computing device is positioned outside of the playing environment. The computing device comprises means for receiving the real-time vital sign signal from the monitoring device, and means for processing the real-time vital sign signal for transmission to and image on the electro-optical display.

Yet another aspect of the present invention is a monitoring device for monitoring the health of a user. The monitoring device includes an article to be worn on the user's wrist, arm or ankle. The article comprises an annular body having an interior surface and an exterior surface. The monitoring device also includes an optical sensor, a circuitry assembly, a display member and a control component. The optical sensor is disposed on the interior surface of the article. The circuitry assembly is preferably embedded within the annular body of the article. The display member is preferably attached to an exterior surface of the annular body of the article. The control component is disposed on the exterior surface of the annular body of the article. The control component controls the input of information and the output of information displayed on the display member.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is a perspective view of a preferred embodiment of a monitoring device worn by a user.

FIG. 1A is a cross-sectional view of the monitoring device along line A-A of FIG. 1.

FIG. 2 is a perspective view of an alternative embodiment of a monitoring device worn by a user.

FIG. 2A is an isolated view of a light source and plurality of photodetectors of the monitoring device of FIG. 2.

FIG. 3 is a systematic diagram of a method of the present invention.

FIG. 3A is an isolated view of a monitoring device of the present invention on a golfer's arm.

FIG. 3B is a cross-sectional view of the monitoring device on the golfer's arm.

FIG. 4 is a cut-away of a user's wrist to illustrate the user's radial artery.

FIG. 5 is a cut-away of a user's ankle to illustrate the user's posterior tibial artery.

FIG. 6 is a schematic diagram of combined circuit assembly and display member utilized with the monitoring device.

FIG. 7 is an isolated side view of a control component utilized with a monitoring device.

FIG. 8 is an isolated top plan view of the control component of FIG. 7.

FIG. 9 is a flow chart for using the control component to input information and output information on a display of the monitoring device.

FIG. 10 is a flow chart of a method of monitoring.

FIG. 11 is an image of an activity log of information obtained from a monitoring device.

FIG. 12 is an image of calorie information obtained from a monitoring device.

### DETAILED DESCRIPTION OF THE INVENTION

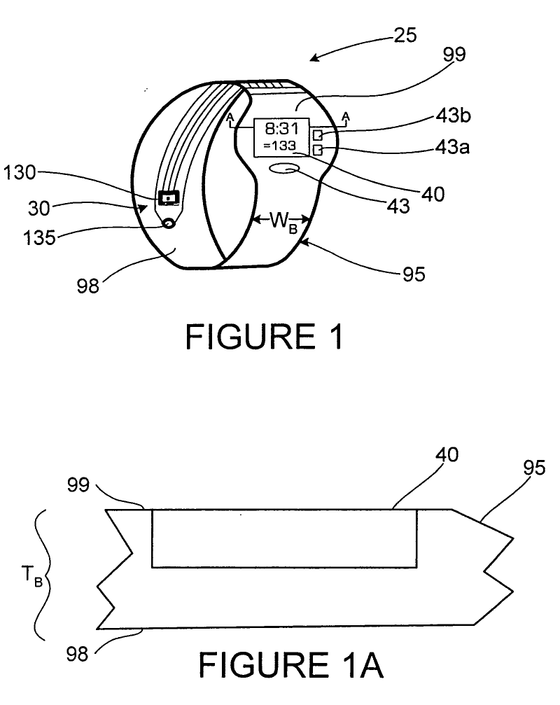

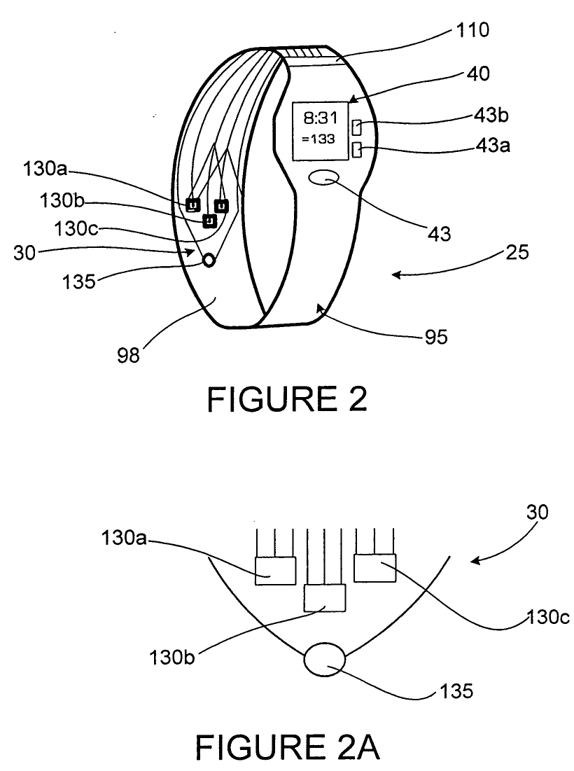

As shown in FIGS. 1, 1A, 2 and 2A, a monitoring device is generally designated 20. The monitoring device 20 preferably includes an article 25, an optical sensor 30, a circuitry assembly 35, a control component 43, connection wires 45 and optionally a display member 40. The monitoring device 20 is preferably worn on a user's wrist 71, arm 72 or ankle 73. The article 25 preferably has a body portion 95 that is sized to securely attach to a user's wrist 71, arm 72 or ankle 73, and the body portion has an interior surface 98 and an exterior surface 99.

It is desirous to adapt the article 25 to the anatomy of the user's wrist 71, arm 72 or ankle 73. The article 25 is preferably composed of neoprene, leather, synthetic leather, LYCRA, another similar material, or a combination thereof. The article 25 preferably has a mass ranging from 5 grams to 50 grams. Preferably, the lower the mass of the article 25, the more comfort to the user.

The optical sensor 30 is preferably positioned on the interior surface 98 of the body portion 95 and connected to the circuitry assembly 35 by the connection wires 45. The connection wires 45 are preferably embedded within the body portion 95.

The optical sensor 30 of the monitoring device 20 is preferably positioned over the radial artery 77 of a user if the article 25 is worn on the user's wrist 71 or arm 72. The optical sensor 30 of the monitoring device 20 is preferably positioned over the posterior tibial artery 79 of a user if the article 25 is worn on the user's ankle 73. However, those skilled in the pertinent art will recognize that the optical sensor may be placed over other arteries of the user without departing from the scope and spirit of the present invention. Further, the optical sensor 30 need only be in proximity to an artery of the user in order to obtain a reading or signal.

In a preferred embodiment, the optical sensor 30 is a photodetector 130 and a single light emitting diode (“LED”) 135 transmitting light at a wavelength of approximately 660 nanometers. As the heart pumps blood through the arteries in the user's ankle or wrist, blood cells absorb and transmit varying amounts of the light depending on how much oxygen binds to the cells' hemoglobin. The photodetector 30, which is typically a photodiode, detects transmission at the red wavelengths, and in response generates a radiation-induced signal.

Alternatively, the optical sensor 30 is a pulse oximetry device with a light source 135 that typically includes LEDs that generate both red (λ˜660 nm) and infrared (λ˜900 nm) radiation. As the heart pumps blood through the arteries in the ankle or wrist of the user, blood cells absorb and transmit varying amounts of the red and infrared radiation depending on how much oxygen binds to the cells' hemoglobin. The photodetector 130, which is typically a photodiode, detects transmission at the red and infrared wavelengths, and in response generates a radiation-induced signal.

Alternatively, the optical sensor 30 is pulse oximetry device comprising the photo-detector 130, a first light source 135 and a second light source 135a, not shown. In this embodiment, the first light source 135 emits light in an infrared range (λ˜900 nm) and the second light source 135a emits light in a red range (λ˜630 nm). Yet in an alternative embodiment, the optical device 30 is based on green light wherein a LED generates green light (λ˜500-600nm), and the phtotodetector detects the green light.

The light source 135 typically is a light-emitting diode that emits light in a range from 550 nanometers to 1100 nanometers. As the heart pumps blood through the patient's finger, blood cells absorb and transmit varying amounts of light (typically in the red, infrared or green light range) depending on how much oxygen binds to the cells' hemoglobin. The photodetector 30, which is typically a photodiode, detects transmission at the red and infrared wavelengths, or green wavelength, and in response generates a radiation-induced current that travels through the connection wires 45 to the circuitry assembly 35 on the article 25.

Alternatively, as shown in FIGS. 2 and 2A, the optical sensor includes a plurality of photodetectors 130 and a single LED 135.

A preferred photodetector 130 is a light-to-voltage photodetector such as the TSL260R and TSL261, TSL261R photodetectors available from TAOS, Inc of Plano Texas. Alternatively, the photodetector 130 is a light-to-frequency photodetector such as the TSL245R, which is also available from TAOS, Inc. The light-to-voltage photodetectors have an integrated transimpedance amplifier on a single monolithic integrated circuit, which reduces the need for ambient light filtering. The TSL261 photodetector preferably operates at a wavelength greater than 750 nanometers, and optimally at 940 nanometers, which would preferably have a LED that radiates light at those wavelengths. A preferred optical sensor 30 utilizing green light is a TRS1755 sensor from TAOS, Inc of Plano Texas. The TRS1755 comprises a green LED light source (567 nm wavelength) and a light-to-voltage converter. The output voltage is directly proportional to the reflected light intensity.

In a preferred embodiment, the circuit assembly 35 is flexible to allow for the contour of the user's wrist or ankle, and the movement thereof. Preferably the dimensions of a board of the circuit assembly 35 are approximately 39 millimeters (length) by approximately 21 millimeters (width) by 0.5 millimeters (thickness).

Alternatively, the circuitry assembly 35 includes a flexible microprocessor board and a flexible pulse oximetry board. An alternative pulse oximetry board is a BCI MICRO POWER oximetry board, which is a low power, micro-size easily integrated board which provides blood oxygenation level, pulse rate (heart rate), signal strength bargraph, plethysmogram and status bits data. The size of the board is preferably 25.4 millimeters (length)×12.7 millimeters (width)×5 millimeters (thickness). The microprocessor board receives data from the pulse oximetry board and processes the data to display on the display member 40. The microprocessor can also store data. The microprocessor can process the data to display pulse rate, blood oxygenation levels, calories expended by the user of a pre-set time period, target zone activity, time and dynamic blood pressure. Alternatively, the circuitry assembly 35 is a single board with a pulse oximetry circuit and a microprocessor.

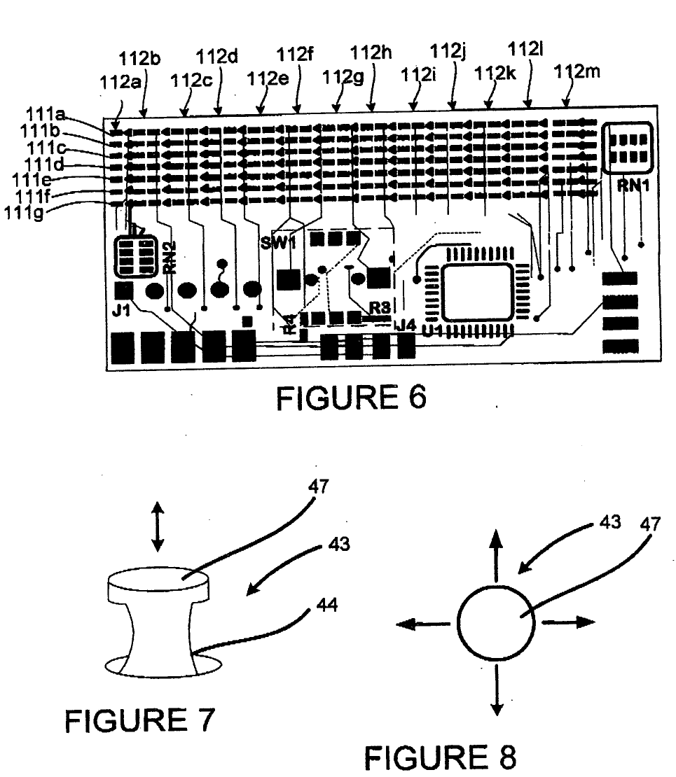

The optional display member 40 is preferably a light emitting diode (“LED”). Alternatively, the display member 40 is a liquid crystal display (“LCD”) or other similar display device. As shown in FIG. 6, the display member 40 is an LED array which preferably has seven rows 111a-111g and thirteen columns 112a-112r. The LED array allows for each column to be illuminated separately thereby giving the appearance of a moving display. For example, if the term “200 calories expended” is displayed on the display member 40, the “2” of the “200” would preferably first appear in column 112m and then subsequently in each of the other columns 112l-112a, from the right-most column to the left-most column thereby giving the appearance of the term scrolling along the display member 40. The terms or words alternatively scroll from left to right. Still alternatively, all of the columns are illuminated at once or all flash in strobe like manner. Those skilled in the pertinent art will recognize alternative methods of displaying information on the display member 40 without departing from the scope and spirit of the present invention.

As shown in FIG. 6, the display member 40 is preferably combined with the circuit assembly 35. A microcontroller 41 processes the signal generated from the optical sensor 30 to generate the plurality of vital sign information for the user which is displayed on the display member 40. The control component 43 is connected to the circuit assembly 35 to control the input of information and the output of information displayed on the display member 40.

FIGS. 7-8 illustrate an isolated view of a preferred embodiment of the control component 43. The control component 43 preferably has a body 44 with a top 47. The body 44 preferably has a shape which minimizes mass and is easily operated by the user. The control component 43 is preferably a button or “joystick” that is capable of multiple dimensional movement such as being compressible up and down as indicated by the arrow in FIG. 7 or in an X-Y movement as indicated by the arrows in FIG. 8. The multiple dimensional movement of the control component 43 allows for the user to enter or select functions and scroll through menus which are displayed on the display member 40, as discussed below. Alternatively, the control component is a plurality of buttons, preferably three, located on the exterior surface 99 of the article 25.

The monitoring device 20 is preferably powered by a power source 110 positioned on the article 25. Preferably the power source 110 is a battery. The power source 110 is preferably connected to the circuit assembly 35 by positive wire and ground wire, and the ground wire and positive wire are embedded within the article 25. The power source 110 is preferably a lithium ion rechargeable battery such as available from NEC-Tokin. The power source preferably has an accessible port for recharging. The circuit assembly 35 preferably requires 5 volts and draws a current of 20-to 40 milliamps. The power source 110 preferably provides at least 900 milliamp hours of power to the monitoring device 20.

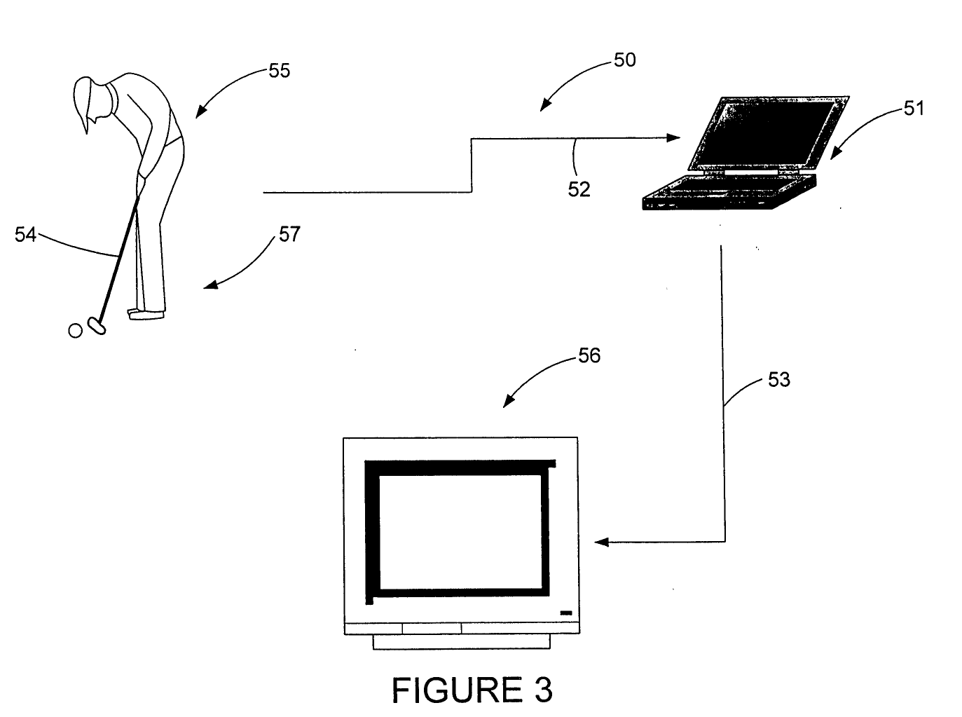

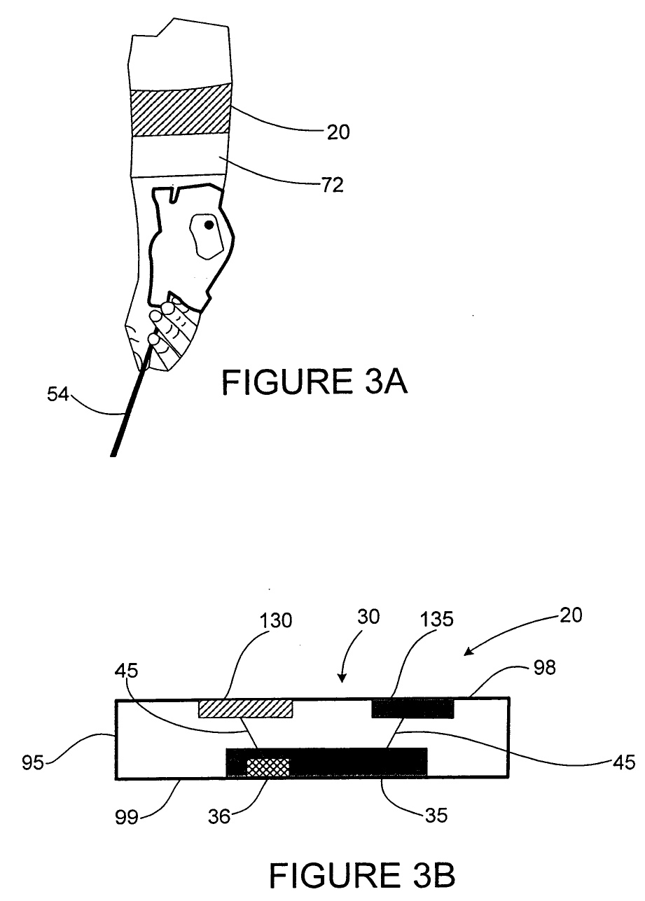

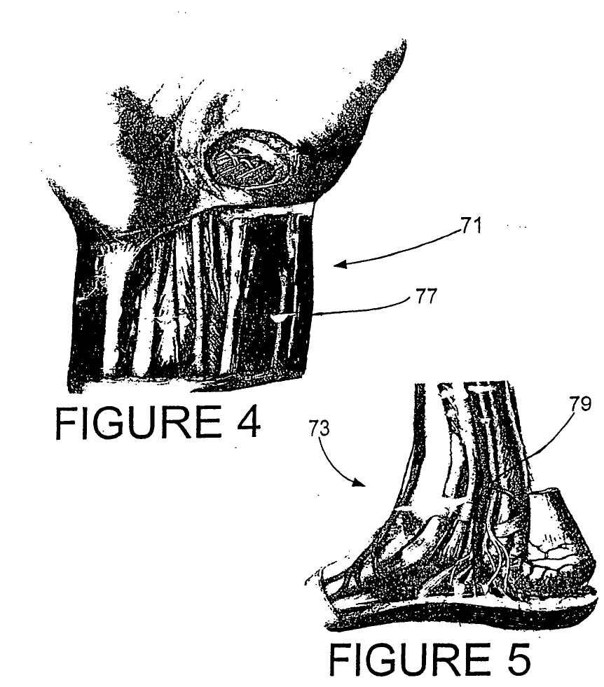

As shown in FIGS. 3, 3A and 3B, a system is generally designated 50. The system 50 preferably includes a monitoring device 20 worn by a user 55, a computing device 51 and an electro-optical display 56. In this embodiment, the monitoring device 20 preferably comprises a short range wireless transceiver 36 which is included in the circuitry assembly 35 for transmitting vital sign data, as shown by first transmission 52, processed from the optical sensor 30 to a handheld device or the computing device 51, preferably positioned outside of a playing environment 57. The display member 40 on the monitoring device 20 is preferably absent in this embodiment. The processed vital sign data is then transmitted, as shown by second transmission 53, to the electro-optical display 56. The electro-optical display 56 may represent a single monitor positioned at the live event for the audience, or it may represent a video broadcast (television broadcast or web broadcast over the internet) for viewers all over the world.

As shown in FIGS. 3 and 3A, the event is a golf match and the user is a golfer. The monitoring device 20 is preferably positioned on an arm 72 of the user 55, preferably over the radial artery. In this manner, the monitoring device 20 can provide a real-time heart rate of the user 55 on the electro-optical display 56 to television viewers. Thus, as the golfer relaxes to putt, viewers can see the golfer's heart rate slow. Alternatively, if the golfer's heart rate does not slow, and the golfer misses the putt, then the television viewers would have real-time facts to provide a reason for missing the putt. The monitoring device is preferably constructed and positioned as to not interfere with the golfer's swing of a club 54.

Alternatively, the monitoring device 20 is worn by a poker player playing in a poker match such as the WORLD SERIES OF POKER™ as set forth at www.worldseriesofpoker.com. In this manner, the television viewer can monitor the heart rate of the poker player as the poker player bets, bluffs, folds, loses or wins. Thus, even though the poker player may appear calm, television viewers can determine by the poker player's heart rate if the poker player is truly calm or bluffing. Other possible applications of the system 50 include tennis players during a tennis match, football players during a football game, boxers during a boxing match, runners during a track event, and the like.

The short-range wireless transceiver 36b is preferably a transmitter operating on a wireless protocol, e.g. Bluetooth™, part-15, or 802.11. “Part-15” refers to a conventional low-power, short-range wireless protocol, such as that used in cordless telephones. The short-range wireless transmitter (e.g., a Bluetooth™ transmitter) receives information from the microprocessor and transmits this information in the form of a packet through an antenna. The external laptop computer 51 or hand-held device features a similar antenna coupled to a matched wireless, short-range receiver that receives the packet. In certain embodiments, the hand-held device is a cellular telephone with a Bluetooth circuit integrated directly into a chipset used in the cellular telephone. In this case, the cellular telephone may include a software application that receives, processes, and displays the information. The secondary wireless component may also include a long-range wireless transmitter that transmits information over a terrestrial, satellite, or 802.11-based wireless network. Suitable networks include those operating at least one of the following protocols: CDMA, GSM, GPRS, Mobitex, DataTac, iDEN, and analogs and derivatives thereof Alternatively, the handheld device is a pager or PDA.

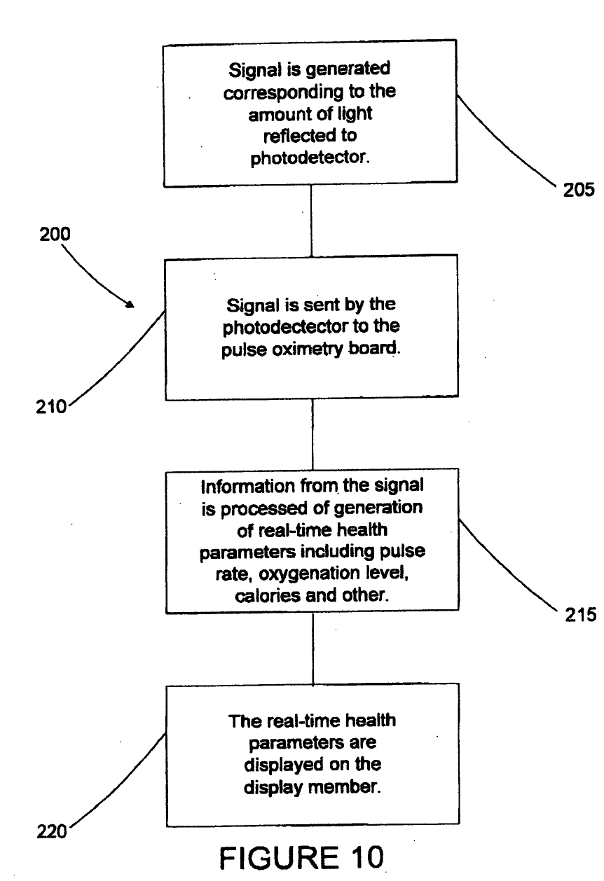

As shown in FIG. 10, a general method is indicated as 200. At block 200, the light source 135 transmits light through or at an artery of the user. The photo-detector 130 detects the light. The pulse rate is determined by the signals received by the photo-detector 130. A ratio of the fluctuation of the red and infrared light signals is used to calculate the blood oxygen saturation level of the user. An optical sensor 30 with a photodetector 130 and single LED 135 is preferably utilized. Alternatively, a pulse oximetry device with two LEDs and a photodetector is utilized.

At block 210, this information is sent to the circuitry assembly 35 for creation of blood oxygenation level, pulse rate, signal strength bargraph, plethysmogram and/or status bits data At block 215, the microprocessor further processes the information to display pulse rate, blood oxygenation levels, calories expended by the user of a pre-set time period, target zones of activity, time and/or dynamic blood pressure. At block 220, the information is displayed on a display member or electro-optical display.

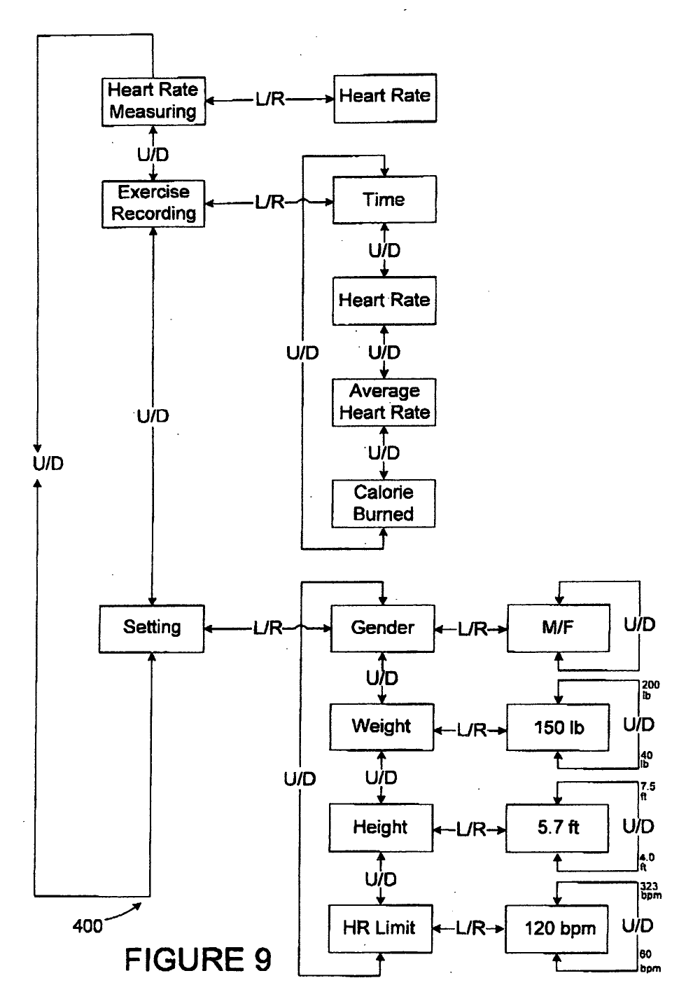

A flow chart diagram 400 for using the control component 43 with the display member 40 is shown in FIG. 9. As mentioned above, the control component 43 allows a user to scroll and select from terms displayed on the display member 40. User inputs preferably include age, gender, weight, height and resting heart rate which can be inputted and stored in a memory of the circuit assembly 35. The real time heart rate of the user is preferably displayed as a default display, and the user's real time heart rate is preferably updated every ten seconds based on measurements from the optical sensor 30. Based on the user inputs, the calories expended by the user for a set time period are calculated and displayed on the display member 40 as desired by the user using the control component 43. The monitoring device 20 will also preferably include a conventional stop watch function, which is displayed on the display member 40 as desired by the user. The display member 40 preferably displays a visual alert when a user enters or exits a target zone such as a cardio zone or fat burning zone. The monitoring device 20 optionally includes an audio alert for entering or exiting such target zones.

The user can toggle the control component 43, or push buttons, to maneuver between the user's real-time heart rate and real time calories expended by the user during a set time period. The user can also scroll through a menu-like display on the display member 40 and enter options by pushing downward on the control component 43. The options can preferably include a “My Data” section which the user inputs by scrolling and selection an option by pushing downward, such as selecting between male and female for gender. The user can also select target zones by scrolling through a different section of the menu. As discussed below, each target zone is calculated using a formula based upon the user's personal data. In operation, when a specific target zone is selected, a visual alert in the form of a specific display such as an icon-like picture is displayed on the display member 40 to demonstrate that the user is now in the specified target zone. The icon preferably blinks for a set period of time such as ten seconds. Those skilled in the pertinent art will recognize that other options may be included on the menu-like display without departing from the spirit and scope of the present invention.

In yet an alternative embodiment, an accelerometer, not shown, is embedded within the main body 95 of the article 25 and connected to the circuitry assembly 35 in order to provide information on the distance traveled by the user. In a preferred embodiment, the accelerometer is a multiple-axis accelerometer, such as the ADXL202 made by Analog Devices of Norwood, Mass. This device is a standard micro-electronic-machine (“MEMs”) module that measures acceleration and deceleration using an array of silicon-based structures.

In yet another embodiment, the monitoring device 20 comprises a first thermistor, not shown, for measuring the temperature of the user's skin and a second thermistor, not shown, for measuring the temperature of the air. The temperature readings are displayed on the display member 40 and the skin temperature is preferably utilized in further determining the calories expended by the user during a set time period. One such commercially available thermistor is sold under the brand LM34 from National Semiconductor of Santa Clara, Calif. A microcontroller that is utilized with the thermistor is sold under the brand name ATMega 8535 by Atmel of San Jose, Calif.

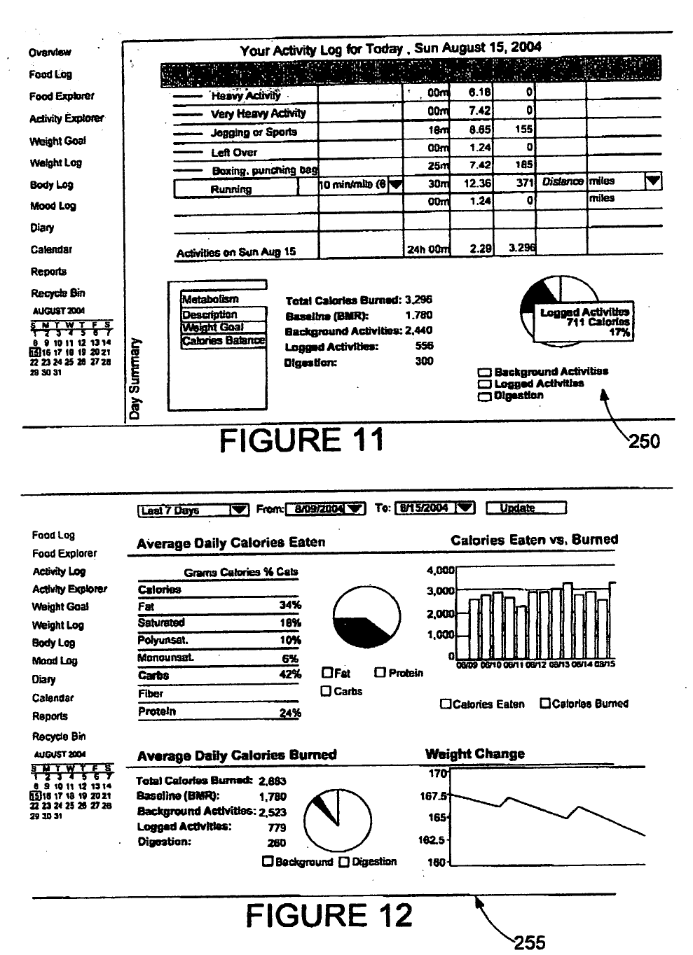

The monitoring device 20 may also be able to download the information to a computer for further processing and storage of information. The download may be wireless or through cable connection. The information can generate an activity log 250 such as shown in FIG. 11, or a calorie chart 255 such as shown in FIG. 12.

The microprocessor can use various methods to calculate calories burned by a user. One such method uses the Harris-Benedict formula. Other methods are set forth at www.unu.edu/unupress/food2/ which relevant parts are hereby incorporated by reference. The Harris-Benedict formula uses the factors of height, weight, age, and sex to determine basal metabolic rate (BMR). This equation is very accurate in all but the extremely muscular (will underestimate calorie needs) and the extremely overweight (will overestimate caloric needs) user.

The equations for men and women are set forth below:
Men:BMR=66+(13.7×mass(kg))+(5×height(cm))−(6.8×age(years))
Women:BMR=655+(9.6×mass)+(1.8×height)−(4.7×age)

The calories burned are calculated by multiplying the BMR by the following appropriate activity factor: sedentary; lightly active; moderately active; very active; and extra active.
Sedentary=BMR multiplied by 1.2 (little or no exercise, desk job)
Lightly active=BMR multiplied by 1.375 (light exercise/sports 1-3 days/wk)
Moderately Active=BMR multiplied by 1.55 (moderate exercise/sports 3-5 days/wk)
Very active=BMR multiplied by 1.725 (hard exercise/sports 6-7 days/wk)
Extra Active=BMR multiplied by 1.9 (hard daily exercise/sports & physical job or 2× day training, marathon, football camp, contest, etc.)

Various target zones may also be calculated by the microprocessor. These target zones include: fat burn zone; cardio zone; moderate activity zone; weight management zone; aerobic zone; anaerobic threshold zone; and red-line zone.
Fat Burn Zone=(220−age)×60% & 70%
An example for a thirty-eight year old female:
(220−38)×0.6=109
(220−38)×0.7=127
Fat Burn Zone between 109 to 127 heart beats per minute.
   Cardio Zone=(220−your age)×70% & 80%
   An example for a thirty-eight year old female:
   (220−38)×0.7=127
   (220−38)×0.8=146
Cardio zone is between 127 & 146 heart beats per minute.

Moderate Activity Zone, at 50 to 60 percent of your maximum heart rate, burns fat more readily than carbohydrates. That is the zone one should exercise at if one wants slow, even conditioning with little pain or strain.

Weight Management Zone, at 60 to 70 percent of maximum, strengthens ones heart and burns sufficient calories to lower one's body weight.

Aerobic Zone, at 70 to 80 percent of maximum, not only strengthens one's heart but also trains one's body to process oxygen more efficiently, improving endurance.

Anaerobic Threshold Zone, at 80 to 90 percent of maximum, improves one's ability to rid one's body of the lactic-acid buildup that leads to muscles ache near one's performance limit. Over time, training in this zone will raise one's limit.

Red-Line Zone, at 90 to 100 percent of maximum, is where serious athletes train when they are striving for speed instead of endurance.

### EXAMPLE ONE

Female, 30 yrs old, height 167.6 centimeters, weight 54.5 kilograms.
The BMR=655+523+302−141=1339 calories/day.
The BMR is 1339 calories per day. The activity level is moderately active (work out 3-4 times per week). The activity factor is 1.55. The TDEE=1.55×1339=2075 calories/day. TDEE is calculated by multiplying the BMR of the user by the activity multiplier of the user.

The heart rate may be used to dynamically determine an activity level and periodically recalculate the calories burned based upon that factor. An example of such an activity level look up table might be as follows:
Activity/Intensity Multiplier Based on Heart Rate
Sedentary=BMR×1.2 (little or no exercise, average heart rate 65-75 bpm or lower)
Lightly active=BMR×3.5 (light exercise, 75 bpm-115 bpm)
Mod. active=BMR×5.75 (moderate exercise, 115-140 pm)
Very active=BMR×9.25 (hard exercise, 140-175 bpm)
Extra active=BMR×13 (175 bpm−maximum heart rate as calculated with MHR formula)

For example, while sitting at a desk, a man in the above example might have a heart rate of between 65 and 75 beats per minute (BPM). (The average heart rate for an adult is between 65 and 75 beats per minute.) Based on this dynamically updated heart rate his activity level might be considered sedentary. If the heart rate remained in this range for 30 minutes, based on the Harris-Benedict formula he would have expended 1.34 calories a minute×1.2 (activity level)×30 minutes, which is equal to 48.24 calories burned.

If the man were to run a mile for 30 minutes, with a heart rate ranging between 120 and 130 bpm, his activity level might be considered very active. His caloric expenditure would be 1.34 calories a minute×9.25 (activity level)×30 minutes, which is equal to 371.85.

Another equation is weight multiplied by time multiplied by an activity factor multiplied by 0.000119.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention:

1. A method of monitoring a plurality of real-time vital signs for a user, the method comprising:
generating a signal corresponding to the flow of blood through an artery of the user, the signal generated from an optical sensor in proximity to the artery of the user, the optical sensor disposed on an article, the article placed on a wrist, arm or ankle of the user;
generating real-time heart rate data of the user and a real-time oxygen saturation level data of the user from the signal generated by the optical sensor;
processing the real-time heart rate data and the real-time oxygen saturation level data of the user for analysis of real-time calories expended by the user and for real-time display of a plurality of the user's vital signs; and
displaying the plurality of user's vital signs on a display.

2. The method according to claim 1 further comprising:
measuring the distance traveled by the user during a time period using an accelerometer disposed on the article, and displaying the distance traveled by the user on the display; and
determining the user's dynamic blood pressure from the heart rate date and the oxygen saturation level of the user, and displaying the user's dynamic blood pressure on the display.

3. The method according to claim 1 further comprising wirelessly transmitting the calories expended by the user, the user's heart rate and the user's blood oxygen saturation level from a wireless transceiver of the article to a mobile communication device or a computer; and wherein the display is an electro-video display.

4. The method according to claim 1 wherein the display is a display member disposed on an exterior surface of the article, the display of the plurality of user's vital signs controlled by the user using a control component disposed on the exterior surface of the article

5. A monitoring device for monitoring at least one vital sign of a user, the monitoring device comprising:
an article having an annular body;
means for measuring blood flow through an artery of a wrist, arm or ankle of the user, the measuring means connected to the article;
means for determining the heart rate of the user based on the blood flow through the artery of the user; and
means for visually displaying the real-time heart rate of the user.

6. The monitoring device according to claim 5 further comprising means for calculating calories expended by the user during a time period and means for displaying the calories expended by the user, the calculating means on the article.

7. The monitoring device according to claim 5 wherein the article has an annular length ranging from 4 inches to 8 inches, a width ranging from 1 inch to 3 inches, and a thickness ranging from 0.10 inch to 1.0 inch, and the article is composed of a material selected from the group consisting of leather, LYCRA and neoprene.

8. The monitoring device according to claim 5 wherein the controlling means is a joystick extending outward from the article, the joystick capable of multiple dimensional movement to input information and control the out of information on the visually displaying means.

9. The monitoring device according to claim 5 further comprising a power source embedded within the annular body of the article, the power source having a port for recharging the power source.

10. The monitoring device according to claim 5 wherein the measuring means is selected from the group consisting of: an optical sensor comprising a light-to-voltage photodetector capable of transmitting a digital signal, and at least one light emitting diode capable of radiating light ranging from 550 nanometers to 1100 nanometers; a pulse oximetry sensor comprising a light-to-voltage photodetector capable of transmitting a digital signal, first light emitting diode capable of radiating red light and a second light emitting diode capable of emitting infrared light; an optical sensor comprising a light-to-frequency photodetector capable of transmitting a digital signal, and at least one light emitting diode capable of radiating light ranging from 600 nanometers to 1100 nanometers; an optical sensor comprising a plurality of light-to-voltage photodetectors capable of transmitting a digital signal; and at least one light emitting diode capable of radiating light ranging from 550 nanometers to 1100 nanometers; an optical sensor comprising a plurality of light-to-frequency photodetectors capable of transmitting a digital signal, and at least one light emitting diode capable of radiating light ranging from 550 nanometers to 1100 nanometers; and an optical sensor comprising a light-to-voltage photodetector capable of transmitting a digital signal, and at least one light emitting diode capable of radiating light at 567 nanometers.

11. The monitoring device according to claim 5 further comprising means for controlling the input information and the output of information displayed on the visually displaying means, the controlling means disposed on the exterior surface of the article.

12. The monitoring device according to claim 5 wherein a plurality of the user's vital signs are displayed on the display member, the plurality of the user's vital signs comprises calories expended by the user, the user's blood oxygen saturation level, a target zone and distance traveled.

13. The monitoring device according to claim 5 utilized in a method for real time monitoring of at least one vital sign of a user during a live event within a playing environment, the method comprising:
obtaining real-time vital sign data from the user during the event, the vital sign data comprising the heart rate of the user;
wirelessly transmitting the vital sign data for user to a transceiver outside of the playing environment; and
processing the vital sign data for the user for display on an electro-optical display device.

14. The method according to claim 13 wherein the monitoring device comprises a short range wireless transmitter for wirelessly transmitting the vital sign data, and wherein the processing of the vital sign date is performed by a computing device.

15. A system for real time monitoring of a user's vital sign during a live event within a playing environment, the system comprising:
a monitoring device attached to an arm, wrist or ankle of the user, the monitoring device comprising
means for generating a real-time vital sign signal corresponding to the heart rate of the user, and
means for transmitting the real-time vital sign signal outside of the playing environment; and
a computing device positioned outside of the playing environment, the computing device comprising
means for receiving the real-time vital sign signal from the monitoring device, and
means for processing the real-time vital sign signal for transmission to and image on an electro-optical display.

16. The system according to claim 15 wherein the generating means of the monitoring device is means is selected from the group consisting of: an optical sensor comprising a light-to-voltage photodetector capable of transmitting a digital signal, and at least one light emitting diode capable of radiating light ranging from 550 nanometers to 1100 nanometers; a pulse oximetry sensor comprising a light-to-voltage photodetector capable of transmitting a digital signal, first light emitting diode capable of radiating red light and a second light emitting diode capable of emitting infrared light; an optical sensor comprising a light-to-frequency photodetector capable of transmitting a digital signal, and at least one light emitting diode capable of radiating light ranging from 600 nanometers to 1100 nanometers; an optical sensor comprising a plurality of light-to-voltage photodetectors capable of transmitting a digital signal, and at least one light emitting diode capable of radiating light ranging from 550 nanometers to 1100 nanometers; an optical sensor comprising a plurality of light-to-frequency photodetectors capable of transmitting a digital signal, and at least one light emitting diode capable of radiating light ranging from 550 nanometers to 1100 nanometers; and an optical sensor comprising a light-to-voltage photodetector capable of transmitting a digital signal, and at least one light emitting diode capable of radiating light at 567 nanometers.

17. The system according to claim 15 wherein the user and live event are selected from the group consisting of: golfer and golf match; poker player and poker match; tennis player and tennis match; boxer and boxing match; football player and football game; and runner and track event.

18. The system according to claim 15 wherein the electro-optical display is a television.

19. The system according to claim 15 wherein the monitoring device further comprises means for generating a plurality of other real-time vital signs for the user, the plurality of other real-time vital signs comprising calories expended, blood oxygen saturation level, distance traveled, and surface body temperature.

20. The system according to claim 15 wherein the transmitting means is a short range wireless transmitter disposed on the monitoring device, and wherein the monitoring device further comprises an article having an annular length ranging from 4 inches to 8 inches, a width ranging from 1 inch to 3 inches, and a thickness ranging from 0.10 inch to 1.0 inch, and the article is composed of a material selected from the group consisting of leather, LYCRA and neoprene.
